(19) United States
(12) Patent Application Publication
(10) Pub. No.: US 2026/0335142 A1
(43) Pub. Date: Oct. 29, 2026

(54) WOUND DRESSING FOR WOUND THERAPY TREATMENT SYSTEM

(71) Applicant: Solventum Intellectual Properties Company, Maplewood, MN (US)

(72) Inventors: Kathleen L. DERRICK, San Antonio, TX (US); Amy K. MCNULTY, Stillwater, MN (US); Bryan A. BAKER, San Antonio, TX (US)

(21) Appl. No.: 19/114,251

(22) PCT Filed: Sep. 19, 2023
(86) PCT No.: PCT/IB2023/059268

§ 371 Date: Mar. 21, 2025

Related U.S. Application Data
(60) Provisional application No. 63/413,684, filed on Oct. 6, 2022.

(51) Int. Cl. A61F 13/01; A61F 13/00; B32B 5/02; B32B 5/18; B32B 5/24; B32B 5/26; D03D 9/00; D03D 15/283; D03D 27/06

(52) U.S. Cl. CPC A61F 13/01029; A61F 13/00987; A61F 13/01017; B32B 5/024; B32B 5/028; B32B 5/18; B32B 5/245; B32B 5/262; D03D 9/00; D03D 15/283; D03D 27/06; B32B 2262/0276; B32B 2266/06; B32B 2307/73; B32B 2307/7376; B32B 2535/00; D10B 2331/04; D10B 2509/022

(57) ABSTRACT

A wound dressing for use with a wound therapy treatment system includes a support layer having a first side configured to face a wound bed and a second side opposite to the first side. The wound dressing further includes a plurality of loop piles made from a single loop material. The plurality of loop piles is arranged on the first side of the support layer and not on the second side of the support layer.

### CROSS-REFERENCE TO RELATED APPLICATIONS

This application is a U.S. National Stage Entry of International Application No. PCT/IB2023/059268, filed Sep. 19, 2023, which claims the benefit of priority to U.S. Provisional Application No. 63/413,684, filed on Oct. 6, 2022, which is incorporated herein by reference in its entirety.

### TECHNICAL FIELD

The present disclosure relates generally to a wound dressing and a wound therapy treatment system including the wound dressing.

### BACKGROUND

Negative pressure wound therapy (NPWT) systems are embodied as sealed wound-care systems particularly indicated for chronic persistent wounds and/or complicated wounds. Specifically, for promoting wound healing, a pressure that is reduced relative to the surroundings (commonly referred to as “negative pressure”) is applied to the wound. The negative pressure causes mechanical contraction of the wound and removal of exudates, such as, slough, necrotic tissue, microbial load (e.g., bacteria and biofilms) from the wound, thus promoting formation of granulation tissues and accelerating wound healing. The NPWT system typically includes a therapy unit that is in fluid communication with a wound site via a wound dressing. Recent advancements in wound healing with the NPWT system involves application of topical fluids to wounds to work in combination with the NPWT system. However, conventional wound dressings may not be compatible with NPWT systems, may be complicated to use with NPWT systems, may be limited by cost, and/or may not allow efficient removal of exudates from the wound.

### SUMMARY

Generally, the present disclosure relates to a wound dressing, a wound therapy treatment system including the wound dressing, a method of using the wound dressing, and a method of forming the wound dressing.

In a first aspect, the present disclosure provides a wound dressing for use with a wound therapy treatment system. The wound dressing includes a support layer having a first side configured to face a wound bed and a second side opposite to the first side. The wound dressing further includes a plurality of loop piles made from a single loop material. The plurality of loop piles is arranged on the first side of the support layer and not on the second side of the support layer.

In a second aspect, the present disclosure provides a wound therapy treatment system including the wound dressing of the first aspect. The wound therapy treatment system further includes a therapy unit. The therapy unit includes at least one of a negative pressure pump and a fluid instillation pump. At least one of the negative pressure pump and the fluid instillation pump is fluidly coupled to the support layer and is configured to deliver at least one of negative pressure and fluid instillation to the wound bed.

In a third aspect, the present disclosure provides a method. The method includes applying the wound dressing of the first aspect to the wound bed. The method further includes operably connecting a therapy unit to at least a portion of the wound dressing.

In a fourth aspect, the present disclosure provides a method of forming a wound dressing. The method includes providing a support layer having a first side configured to face a wound bed and a second side opposite to the first side. The method further includes forming a plurality of loop piles made from a single loop material on the first side of the support layer and not on the second side of the support layer.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic sectional view of a wound therapy treatment system coupled to a user according to an embodiment of the present disclosure;

FIG. 2 illustrates a schematic sectional view of a wound dressing associated with the wound therapy treatment system of FIG. 1 according to an embodiment of the present disclosure;

FIG. 3 illustrates a photograph of the wound dressing of FIG. 2;

FIG. 4 illustrates a flowchart for a method of forming the wound dressing according to an embodiment of the present disclosure;

FIG. 5 illustrates a flowchart for a method of using the wound dressing according to an embodiment of the present disclosure;

FIG. 6A illustrates a photograph of a first wound dressing sample disposed in a blood soil model before an instillation and negative therapy cycle according to an embodiment of the present disclosure;

FIG. 6B illustrates a photograph of a second wound dressing sample disposed in the blood soil model before an instillation and negative therapy cycle according to another embodiment of the present disclosure;

FIG. 7A illustrates a photograph of a post-treatment blood soil model in a petri dish after the first wound dressing sample of FIG. 6A was removed from the petri dish after the instillation and negative therapy cycle;

FIG. 7B illustrates a photograph of the post-treatment blood soil model in a petri dish after the second wound dressing sample of FIG. 6B was removed from the petri dish after the instillation and negative therapy cycle; and

FIG. 7C illustrates a photograph of the post-treatment blood soil model in a petri dish after an exemplary wound dressing sample was removed from the petri dish after an instillation and negative therapy cycle.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “same” or “similar” as a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the terms “layer,” “sheet,” and “dressing,” or variations thereof, are used to describe an article having a thickness that is small relative to its length and width.

As used herein, the term “negative pressure” broadly refers to a pressure lower than a local ambient pressure, such as an ambient pressure, in a local environment outside the sealed treatment environment provided by a dressing. In many cases, the local ambient pressure can also be the atmospheric pressure at which a wound site is located. Alternatively, the pressure can be less than the hydrostatic pressure associated with the tissue at the wound site. Unless otherwise specified, the pressure values described herein are gauge pressures. Similarly, a reference to an increase in negative pressure typically refers to a decrease in absolute pressure, while a decrease in negative pressure typically refers to an increase in absolute pressure.

As used herein, the term “wounds” can include, for example, chronic, acute, traumatic, subacute, and dehiscence wounds, partially thick burns, ulcers (such as, diabetic, compressive, or venous insufficiency ulcers), flaps, and grafts.

As used herein, the term “wound bed”, may include a wound site of a tissue site, such as, bone tissue, adipose tissue, muscle tissue, nerve tissue, skin tissue, vascular tissue, connective tissue, cartilage, tendons, or ligaments. The term “wound bed” may also refer to an area of a tissue that is not necessarily a wound or defect but may be desired to add or promote additional tissue growth. For example, negative pressure can be used in a particular tissue area to grow additional tissue that can be harvested or transplanted to another tissue site.

Conventionally, a negative pressure wound therapy system (NPWT) system is used to promote wound healing. The NPWT system includes a therapy device configured to provide negative pressure wound therapy by reducing a pressure at a wound. The therapy device can draw a vacuum (relative to atmospheric pressure) at the wound by removing wound exudate, air, and other fluids from the wound. The fluids removed from the wound may be collected within an exudate canister. The therapy device may also extract exudates from the wound that may be previously delivered to the wound. The instillation fluid can include, for example, a cleansing fluid, a prescribed fluid, a medicated fluid, an antibiotic fluid, or any other type of fluid which can be delivered to the wound during wound treatment. The instillation fluid may be held in an instillation canister and controllably dispensed to the wound via a tubing. Further, the NPWT system includes a wound dressing that is placed at the wound and connected in fluid communication with the instillation canister and the exudate canister.

Thus, the NPWT system, including the wound dressing, may be used for wound healing. However, conventional wound dressings may be complex to use with NPWT systems, may be limited by cost, and/or may not allow efficient wound cleaning and healing.

The present disclosure provides a wound dressing for use with a wound therapy treatment system. The wound dressing includes a support layer having a first side configured to face a wound bed and a second side opposite to the first side. The wound dressing further includes a plurality of loop piles made from a single loop material. The plurality of loop piles is arranged on the first side of the support layer and not on the second side of the support layer.

The loop piles of the wound dressing may attach to exudates, such as, slough and may assist in removal of the exudates out of the wound bed. The wound dressing described herein may exhibit enhanced abrasive properties on the first side thereof which may allow breakdown of thick, highly viscous slough and/or devitalized tissues. Thus, the wound dressing may further improve removal of more adherent slough and/or may reduce time required for adequate debridement. Overall, the wound dressing described herein may be compatible with NPWT systems, may be easy to use with NPWT systems, may be cost-effective, and may allow efficient removal of exudates from the wound bed. The present disclosure provides for simple, inexpensive, and effective debridement that can be performed by caregivers or patients in-home or at clinics.

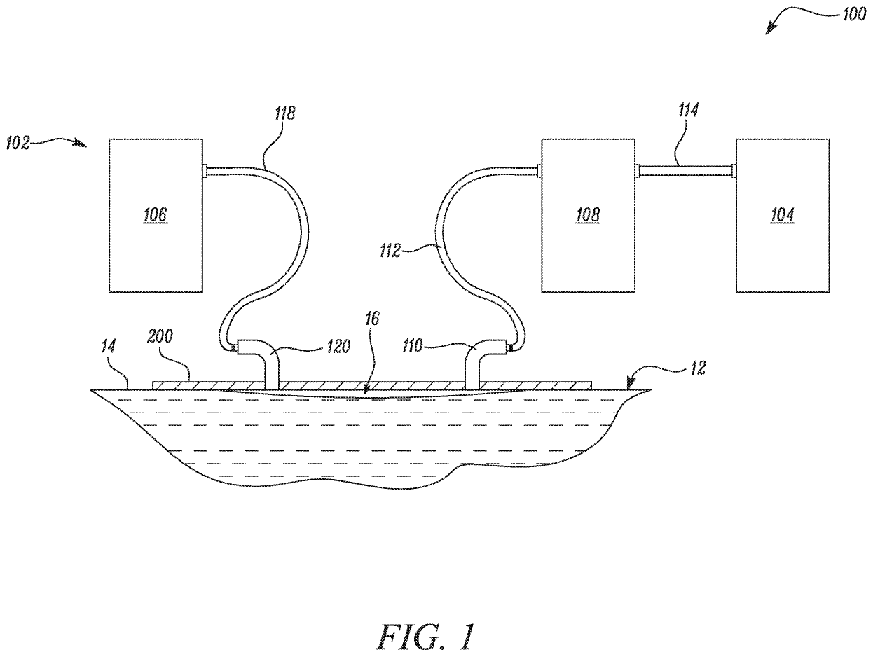

Referring now to Figures, FIG. 1 illustrates a schematic sectional view of a wound therapy treatment system 100 according to an embodiment of the present disclosure. The wound therapy treatment system 100 is used to heal a wound of a user 12. A skin 14 of the user 12 includes a wound bed 16 that is to be treated. The wound therapy treatment system 100 may be used to provide negative pressure therapy, instillation of topical treatment solutions, and debridement in accordance with this specification. The wound therapy treatment system 100 includes a wound dressing 200 and a therapy unit 102. The wound dressing 200 may be positioned at the wound bed 16. The therapy unit 102 includes at least one of a negative pressure pump 104 and a fluid instillation pump 106. In the illustrated embodiment of FIG. 1, the therapy unit 102 includes each of the negative pressure pump 104 and the fluid instillation pump 106. In other embodiments, the therapy unit 102 may omit the fluid instillation pump 106.

Further, the negative pressure pump 104 may include a manual or an electrically powered device that can reduce pressure in a sealed volume, such as, a vacuum pump, a suction pump, a wall suction port available at many healthcare facilities, or a micro-pump, for example. The negative pressure pump 104 may be housed within or used in conjunction with other components, such as, sensors, processing units, alarm indicators, a memory, databases, software, display devices, or user interfaces that may further facilitate the negative pressure therapy. The therapy unit 102 may also include an exudate container 108, coupled to the wound dressing 200 and to the negative pressure pump 104. Negative pressure applied at the wound bed 16 can induce macrostrain and microstrain at the wound bed 16, as well as remove exudates and other fluids from the wound bed 16, which can be collected in the exudate container 108 and discarded in an appropriate manner. In some embodiments, the exudate container 108 may be fluidly coupled to the wound dressing 200 by a connector 110 and a tube 112. Further, the exudate container 108 may be fluidly coupled to the negative pressure pump 104 by a tube 114. In one exemplary embodiment, the connector 110 may allow the negative pressure generated by the negative pressure pump 104 to be delivered to the wound bed 16.

Further, the fluid instillation pump 106 may be fluidly coupled to the wound dressing 200 by a tube 118 and a connector 120 for fluid instillation, as illustrated in FIG. 1. The tube 118 and the connector 120 may allow fluid to be delivered to the wound bed 16. The fluid instillation pump 106 may receive and direct instillation fluid from an instillation fluid source (not shown) towards the wound bed 16. In some embodiments, the fluid instillation pump 106 may include a peristaltic pump. Further, the instillation fluid may include, for example, a cleansing fluid, a prescribed fluid, a medicated fluid, an antibiotic fluid, or any other type of fluid which can be delivered to the wound bed 16 during wound treatment.

In general, components of the therapy unit 102 may be coupled directly or indirectly. For example, the negative pressure pump 104 may be directly coupled to the exudate container 108 and indirectly coupled to the wound dressing 200 through the exudate container 108. Components may be fluidly coupled to each other to provide a path for transferring fluids (i.e., liquid and/or gas) between the components.

In some embodiments, the components of the therapy unit 102 may be fluidly coupled through a tube, such as, the tube 112, the tube 114, and the tube 118. A “tube”, as used herein, broadly refers to a tube, pipe, hose, conduit, or other structure with one or more lumina adapted to convey a fluid between two ends. In some embodiments, components may additionally or alternatively be coupled by virtue of physical proximity, being integral to a single structure, or being formed from the same piece of material. A “connector”, such as, the connector 110 and the connector 120, may be used to fluidly couple a tube to a sealed therapeutic environment. In one illustrative embodiment, each of the connectors 110, 120 may be a T.R.A.C.® Pad or Sensa T.R.A.C.® Pad available from KCI of San Antonio, Texas. In other exemplary embodiments, a connector may also be a tube inserted through a drape.

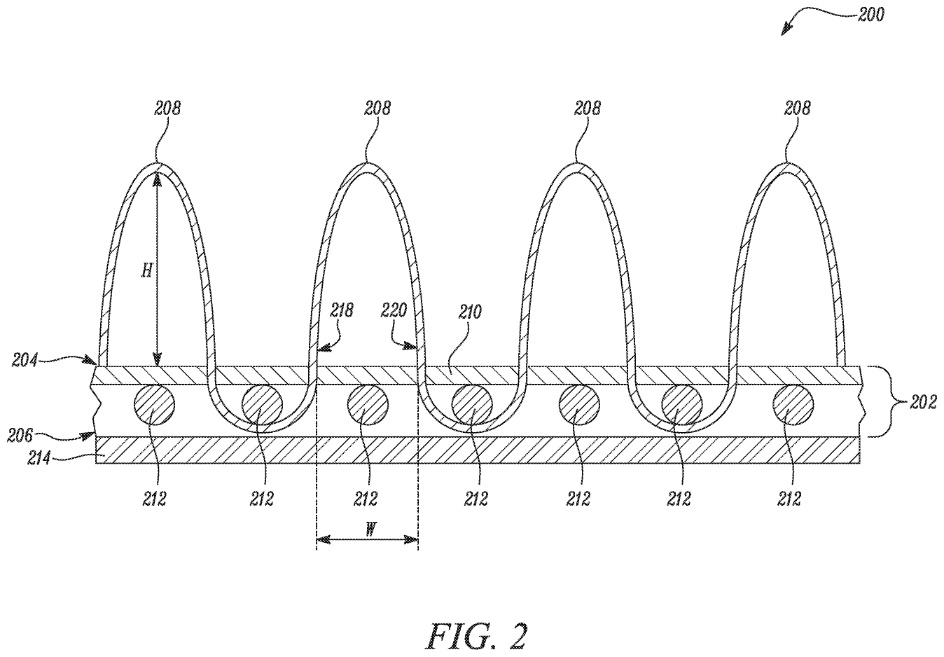

FIG. 2 illustrates a schematic sectional side view of the wound dressing 200 for use with the therapy unit 102 (see FIG. 1). The wound dressing 200 includes a support layer 202 having a first side 204 configured to face the wound bed 16 (see FIG. 1) and a second side 206 opposite to the first side 204. Further, at least one of the negative pressure pump 104 (see FIG. 1) and the fluid instillation pump 106 (see FIG. 1) is fluidly coupled to the support layer 202 and is configured to deliver at least one of negative pressure and fluid instillation to the wound bed 16.

The wound dressing 200 further includes a plurality of loop piles 208 made from a single loop material. The plurality of loop piles 208 is arranged on the first side 204 of the support layer 202 and not on the second side 206 of the support layer 202. The plurality of loop piles 208 may be configured to loosen and retain debrided wound tissues to remove debrided wound tissues from the wound bed 16, such as slough (e.g., necrotic tissue, wet necrotic tissue, dry necrotic tissue, and/or fibrotic tissue). In some embodiments, the plurality of loop piles 208 of the wound dressing 200 in conjunction with the therapy unit 102 may debride and remove at least 80% of fluids and exudates from the wound bed 16.

The support layer 202 is configured to provide a structure to which yarn or threads may be fixed by, e.g., knitting, knotting, weaving, adhering, or the like. In some embodiments, the support layer 202 includes 500 deniers and 840 deniers multifilament natural polyester warp fibers 210 and 2000 deniers multifilament natural polyester weft fibers 212. In some embodiments, the support layer 202 includes a scrim. In some embodiments, the plurality of loop piles 208 are woven into the scrim. In some examples, the support layer 202 may include woven or nonwoven fibers, threads, or yarns defining the scrim. The scrim may include a coarse-woven fabric and/or gauze.

The support layer 202 may include any suitable material or combination of materials. Exemplary materials may include one or more of olefins, polyesters, natural polyesters, rayon, cotton, combinations thereof, or the like. In some examples, the material of the support layer 202 may be selected to maintain structural integrity during a sterilization procedure using, for example, steam, ethylene oxide, dry heat, hydrogen peroxide vapor, gamma radiation, e-beam, or one or more other sterilants.

In some embodiments, the support layer 202 may include a biocompatible thermoset resin including an abrasive material. Specifically, the first side 204 of the support layer 202 may include the biocompatible thermoset resin including the abrasive material. In some embodiments, the abrasive material includes at least one of aluminum oxide, titanium dioxide, and cured resin.

In some embodiments, the wound dressing 200 includes at least one absorbent layer 214 adjacent to the support layer 202. The at least one absorbent layer 214 may be configured to absorb fluids, biological fluids, blood, plasma, saline, water, or other fluids used or produced during wound debridement. In such embodiments, the wound dressing 200 may include a two-part construction. In other words, the wound dressing 200 includes the support layer 202 and the at least one absorbent layer 214 coupled to the support layer 202. In the illustrated embodiment of FIG. 2, the wound dressing 200 includes a single absorbent layer 214. Alternatively, the wound dressing 200 may include two or more absorbent layers, similar to the absorbent layer 214. The at least absorbent layer 214 is disposed at the second side 206 of the support layer 202.

In some embodiments, the at least one absorbent layer 214 includes a hydrophobic, open-cell foam. The at least one absorbent layer 214 may include any other suitable material or combination of materials configured to absorb fluids, such as, for example, cellulose fibers, cotton, water-absorbent polymers, hydrogels, or other materials configured to absorb aqueous solutions. In an example, the at least one absorbent layer 214 may include GranuFoam® material available from Kinetic Concepts, Inc. GranuFoam® may provide uniform distribution of negative pressure at the wound bed 16. Moreover, a hydrophobic construction of GranuFoam® may help in easier removal of exudates. In some examples, the at least one absorbent layer 214 may be ultrasonically welded to the support layer 202.

In some examples, the wound dressing 200 may include an antimicrobial (not shown). The antimicrobial may be disposed on or within any one of the support layer 102 or any other layer of the wound dressing 200, or within an antimicrobial layer that is separate from other layers of the wound dressing 200. The antimicrobial may include any suitable antimicrobial for use on wounds that require debridement. For example, the antimicrobial may include one or more of iodine; iodophors; N-vinyl caprolactam containing polymers; chlorhexidine salts; octenidine salts; parachlorometaxylenol (PCMX); triclosan; hexachlorophene; fatty acid monoesters of glycerin and propylene glycol such as glycerol monolaurate, glycerol monocaprylate, glycerol monocaprate, propylene glycol monolaurate, propylene glycol monocaprylate, propylene glycol monocaprate; phenols; surfactants and polymers that include a C12-C22 hydrophobe and a quaternary ammonium group; polyquatemary amines such as polyhexamethylene biguanide; quaternary silanes; hydrogen peroxide; silver and silver salts such as silver chloride, silver oxide and silver sulfadiazine; or other antimicrobials configured for use on wounds.

In some embodiments, the support layer 202 includes a hydrophobic, open-cell foam. In other words, the support layer 202 may be made of the hydrophobic, open-cell foam instead of the scrim. In such embodiments, the wound dressing 200 may include an all-in-one/unitary design. Further, in such embodiments, the wound dressing 200 may omit the at least one absorbent layer 214. In an example, the support layer 202 may include GranuFoam®. GranuFoam® may provide uniform distribution of negative pressure at the wound bed 16. Moreover, a hydrophobic construction of Granulomata help in easier removal of exudates.

In some embodiments, the wound dressing 200 is devoid of any other loop piles other than the plurality of loop piles 208. In an example, the plurality of loop piles 208 are formed by knitting the single loop material to the first side 204 of the support layer 202. In other examples, the loop piles 208 may be formed by knotting, weaving, adhering, or otherwise fixing the single loop material with the support layer 202. In some examples, the plurality of loop piles 208 may be formed by coupling the single loop material with at least one of the warp fibers 210 or the weft fibers 212 of the support layer 202. The loop piles 208 may include any suitable construction, shape, and/or density.

Further, the single loop material of the plurality of loop piles 208 may include any suitable material or combination of materials. Exemplary single loop materials may include one or more of olefins, polyesters, natural polyesters, acrylic, high density polyethylene (HDPE), polyethylene terephthalate (PET), fluorinated ethylene propylene (FEP), rayon, cotton, combinations thereof, or the like. In some examples, the single loop material may be selected to maintain structural integrity during a sterilization procedure using, for example, steam, ethylene oxide, dry heat, hydrogen peroxide vapor, gamma radiation, or one or more other sterilant.

In some embodiments, the single loop material includes textured natural polyester yarn. In some examples, the single loop material may include 225 denier textured natural polyester yarn. In some embodiments, the single loop material includes bi-component black polyester monofilament yarn.

In some embodiments, a linear mass density of the single loop material is within a range from about 50 deniers to about 2000 deniers. Alternatively, the single loop material may include any other suitable linear mass density. In an example, for relatively softer loop piles, the linear mass density of the single loop material is within a range from about 50 deniers to about 500 deniers, such as, between about 100 deniers and 300 deniers. Further, for relatively stiffer loop piles, the linear mass density of the single loop material may be within a range from about 1000 deniers to about 2000 deniers, such as between about 1200 deniers and 1800 deniers.

In some embodiments, each loop pile 208 of the plurality of loop piles 208 includes about 4 ends of yarn. In other embodiments, each loop pile 208 of the plurality of loop piles 208 may include any suitable number of yarns. The yarns may be braided or unbraided. Multiple yarns in each loop pile 208 may, in some examples, improve an ability of the loop piles 208 to loosen and/or retain debrided wound tissues to remove debrided wound tissues from the wound bed 16. In some examples, the yarns may define a chenille yarn including, for example, a loop pile 208 between two core yarns that are twisted together.

In some embodiments, an average loop height H relative to the support layer 202 of the plurality of loop piles 208 is within a range from about 5 millimeters (mm) to about 10 mm. In other embodiments, the average loop height H may vary as per application requirements. In some examples, the average loop height H of each loop pile 208 of the plurality of loop piles 208 may be substantially similar, e.g., within common tolerances of textile manufacturing techniques. In some examples, the average loop heights of different portions of the loop piles 208 may regularly or irregularly vary. For example, a first portion of the loop piles 208 may include a first average height and a second portion of the loop piles 208 may include a second average height different than the first average height. The selected average loop height H of the loop piles 208 may, in some examples, improve an ability of the loop piles 208 to loosen and/or retain debrided wound tissues to remove debrided wound tissues from the wound bed 16. For example, a relatively shorter loop pile may offer improved debridement, e.g., loosening of tissues to be debrided, compared to a relatively longer loop pile. A relatively longer loop pile may have improved debrided tissue retention, e.g., able to retain a larger volume of debrided wound tissues to remove debrided wound tissues from the wound bed 16, compared to a relatively shorter loop pile.

In some embodiments, an average loop width W between opposing portions 218, 220 of the plurality of loop piles 208 measured parallel to the support layer 202 is within a range from about 0.5 mm to about 10 mm. In other embodiments, the average loop width W may vary as per application requirements.

In some examples, the average loop width W of the plurality of loop piles 208 may be substantially similar, e.g., within common tolerances of textile manufacturing techniques. In some examples, average loop widths of different portions of the loop piles 208 may regularly or irregularly vary. For example, a first portion of the loop piles 208 may include a first average width and a second portion of loop piles 208 may include a second average width different than the first average width. The selected average loop width W of the loop piles 208 may, in some examples, improve an ability of the loop piles 208 to loosen and/or retain debrided wound tissues to remove debrided wound tissues from the wound bed 16. For example, a relatively thinner loop pile may have improved debridement, e.g., loosening of tissue to be debrided, compared to a relatively wider loop pile. A relatively wider loop pile may have improved debrided tissue retention, e.g., able to retain a larger volume of debrided wound tissues to remove debrided wound tissues from the wound bed 16, compared to a relatively thinner loop pile.

Further, the plurality of loop piles 208 may be arranged on the support layer 202 in any suitable density and/or knitting pattern. In some embodiments, a density of the plurality of loop piles 208 is within a range from about 50 stiches per inch to about 400 stiches per inch. In some examples, the density of the loop piles 208 may be within a range from about 200 stiches per inch to about 300 stiches per inch. In an example, the density of the loop piles 208 may be about 270 stiches per inch. Stich per inch may be equal to the product of a course per inch and a wales per inch. In some embodiments, the course per inch of the plurality of loop piles 208 is within a range from about 5 courses per inch to about 30 courses per inch. In some embodiments, the wales per inch of the plurality of loop piles 208 is within a range from about 5 courses per inch to about 30 courses per inch. In some examples, a selected density of the loop piles 208 may improve an ability of the loop piles 208 to loosen and/or retain debrided wound tissue to remove debrided wound tissues from the wound bed 16. In some examples, a density of the loop piles 208 may be selected based on experimental data, such as data obtained from experimental removal of orange pith, or other experimental data indicative of an ability to loosen and/or retain debrided wound tissues to remove debrided wound tissues from the wound bed 16.

In some examples, the position and/or the number of the plurality of loop piles 208 may be selected to improve debridement of wounds by, for example, enabling improved control of a pressure applied via the wound dressing 200 to the wound and/or a movement of the wound dressing 200 relative to the wound during a debridement procedure.

In some examples, the wound dressing 200 may be included in a kit for debriding a wound. An exemplary kit may include the therapy unit 102 (see FIG. 1), the wound dressing 200, instructions, one or more solutions, one or more instillation fluid sources, one or more moisture absorbent members, personal protective equipment, one or more bandages, or other articles. The instructions may include information related to using the wound dressing 200 and/or performing the negative pressure therapy.

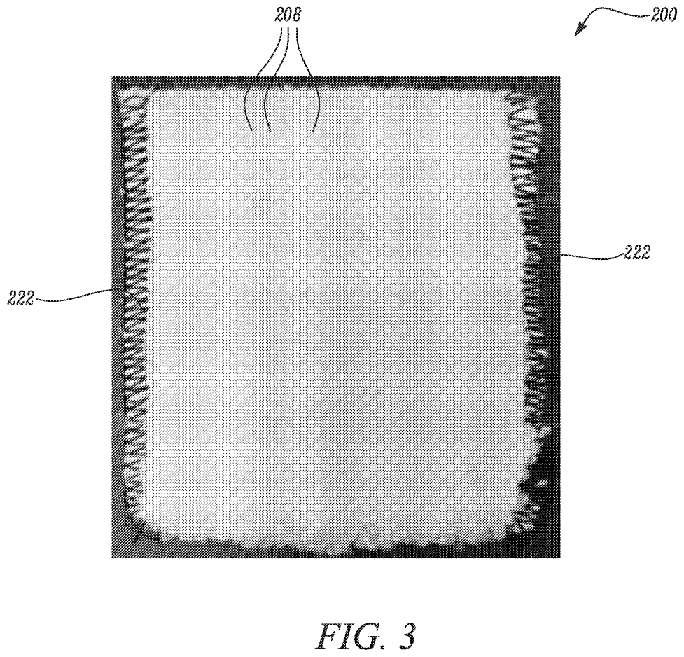

FIG. 3 is a photograph of the wound dressing 200 including the support layer 202 (see FIG. 2) and the plurality of loop piles 208. In an example, at least one free edge 222 of the support layer 202 is secured to prevent yarn fraying. In some embodiments, the at least one free edge 222 of the wound dressing 200 may be secured to itself by impulse welding, ultrasonic welding, and serge seaming. In the illustrated embodiment of FIG. 3, two free edges 222 of the support layer 202 are secured by serge seaming.

The loop piles 208 of the wound dressing 200 may attach to exudates, such as, slough and may allow removal of the exudates from the wound bed 16 (see FIG. 1). The wound dressing 200 described herein may exhibit enhanced abrasive properties on the first side 204 (see FIG. 1) thereof which may allow break down of thick, highly viscous slough, and/or devitalized tissues. Thus, the wound dressing 200 may improve removal of more adherent slough and/or may reduce time required for adequate debridement. Overall, the wound dressing 200 described herein may be compatible with wound therapy treatment systems, and more particularly, negative pressure wound therapy systems. Further, the wound dressing 200 may be easy to use with negative pressure therapy systems, such as, the wound therapy treatment system 100 (see FIG. 1), may be cost-effective, and may allow efficient removal of exudates from the wound bed 16. The present disclosure provides for simple, inexpensive, and effective debridement that can be performed by caregivers or patients in-home or at clinics.

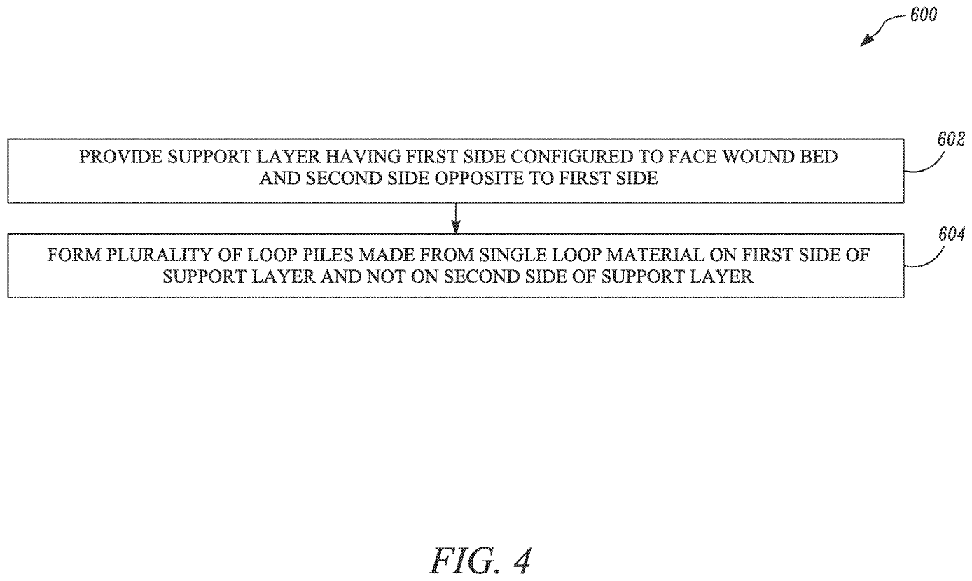

FIG. 4 illustrates a flowchart for a method 600 of forming the wound dressing 200 shown in FIG. 1, according to an embodiment of the present disclosure.

Referring to FIGS. 1-4, at step 602, the method 600 includes providing the support layer 202 having the first side 204 configured to face the wound bed 16 and the second side 206 opposite to the first side 204. At step 604, the method 600 includes forming the plurality of loop piles 208 made from the single loop material on the first side 204 of the support layer 202 and not on the second side 206 of the support layer 202. The wound dressing 200 is devoid of any other loop piles other than the plurality of loop piles 208. In some embodiments, the step of forming the plurality of loop piles 208 further includes knitting the single loop material to the first side 204 of the support layer 202.

In some embodiments, the method 600 further includes a step of securing the at least one free edge 222 of the support layer 202. The securing of the at least one free edge 222 of the support layer 202 includes at least one of impulse welding, ultrasonic welding, and serge seaming. In some embodiments, the method 600 further includes securing the at least one absorbent layer 214 to the support layer 202.

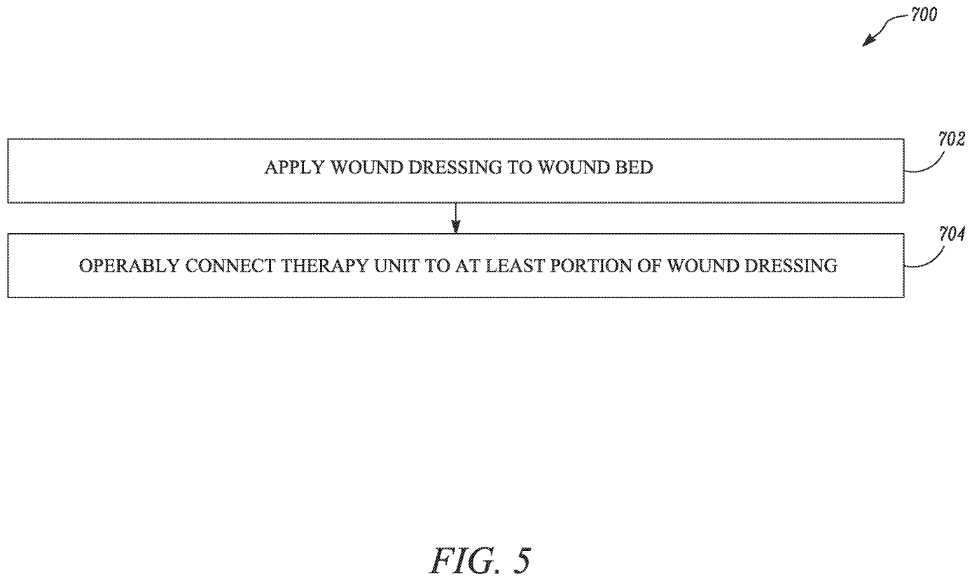

FIG. 5 illustrates a flowchart for a method 700 of using the wound dressing 200 shown in FIG. 1, according to an embodiment of the present disclosure.

Referring to FIGS. 1 and 5, at step 702, the method 700 includes applying the wound dressing 200 to the wound bed 16. At step 704, the method 700 includes operably connecting the therapy unit 102 to at least a portion of the wound dressing 200.

Further, in some embodiments, the step of operably connecting the therapy unit 102 includes fluidly coupling the negative pressure pump 104 to at least the portion of the wound dressing 200. In some embodiments, the method 600 further includes a step of delivering at least one of the negative pressure and the fluid instillation to at least the portion of the wound dressing 200.

### EXAMPLE

The present disclosure is further described with reference to the following example that explains performance of the wound dressing 200 shown in FIG. 1. The example will be explained in reference to FIGS. 1 to 7A-7C.

The following example is merely meant to exemplify the present disclosure, but is not intended to limit or otherwise define the scope of the present disclosure.

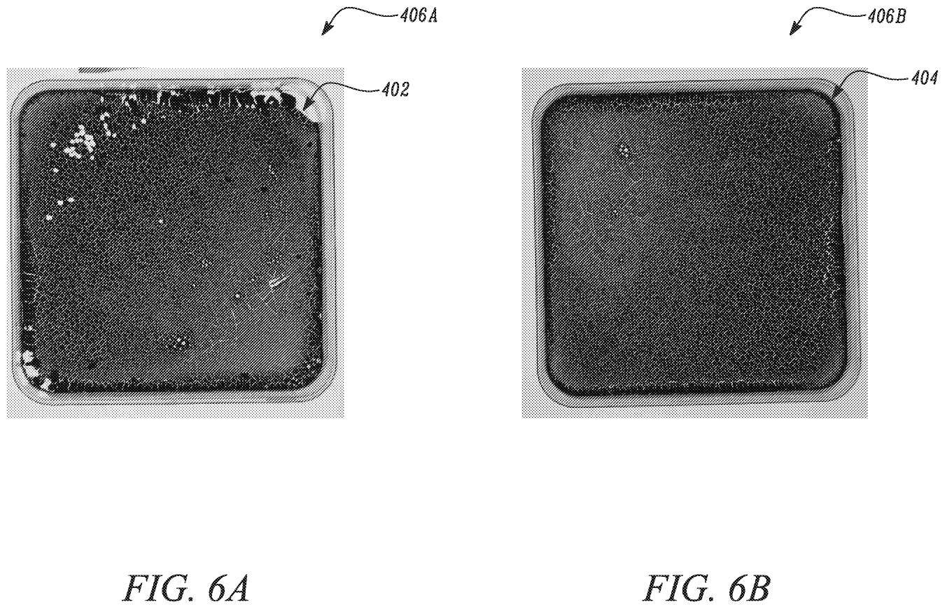

FIGS. 6A and 6B are respective photographs of a first wound dressing sample 402 and a second wound dressing sample 404 disposed in a blood soil model before an instillation and negative therapy cycle was performed thereon. Specifically, the first wound dressing sample 402 is shown disposed in the blood soil model in a petri dish 406A before the instillation and negative therapy cycle was performed thereon and the second wound dressing sample 404 is shown disposed in the blood soil model in a petri dish 406B before the instillation and negative therapy cycle was performed thereon.

The first wound dressing sample 402 included the relatively stiffer loop piles whereas the second wound dressing sample 404 included the relatively softer loop piles. The first and second wound dressing samples 402, 404 were tested to determine a performance of the wound dressing 200 having the single loop material. The first and second wound dressing samples 402, 404 were clotted with hemoglobin/blood clotted models. Specifically, the first and second wound dressing samples 402, 404 were clotted with the blood soil model having a soluble component (hemoglobin and albumin) and an insoluble component (fibrin). For explanatory purposes, steps performed during testing of the first wound dressing sample 402 will be explained in detail. However, the details provided below are equally applicable to the testing of the second wound dressing sample 404.

Details of steps performed during the testing as well as various testing conditions of the first wound dressing sample 402 will now be described in detail.

### Preparation of Blood Soil Model

The blood soil model included bovine serum albumin, hemoglobin from bovine blood, fibrinogen from bovine plasma, and thrombin from bovine plasma. The above mentioned regents were purchased from Millipore-Sigma. The blood soil model was prepared by mixing the reagents in a two-component system as below:

### Component A

1) 4 gram (gm) of Albumin
2) 4 gm of Hemoglobin
3) 600 milligram (mg) of Fibrinogen
4) 50 milliliter (ml) of salt solution containing 142 millimole (mmol) of sodium chloride (NaCl) and 3 mmol of calcium chloride (CaCl₂)

### Component B

1) 4 gm of Albumin
2) 4 gm of Hemoglobin
3) 125 units of Thrombin [actual=2 mg at 133 units per mg=260 U thrombin]
4) 50 ml of salt solution (142 mmol of NaCl and 3 mmol of CaCl₂)

### Mixing Protocol

Component A and component B were mixed together for about 1 hour at room temperature on a bottle roller until the formation of an evenly uniform liquid of dark red coloration. Further, 5 ml of component A and 5 ml of component B were added to the petri dish 406A. The petri dish 406A was a 100 millimeters (mm)×100 mm square petri dish by Fisher Scientific Catalogue #FB0875711A. The petri dish 406A was gently moved side-to-side by hand to ensure even coverage of protein solutions over the dish area. The mixture was set at room temperature for 30 minutes or until set. The blood soil model was then allowed to dehydrate at room temperature overnight. A cover of the petri dish 406A was removed during this time. Further, the blood soil model was stored at 4 °C until use.

### Dressing Setup

The first wound dressing sample 402 was sized by hand approximately 100 mm×100 mm and placed directly on the blood soil model. Dressing components were from V.A.C. VERAFLO Dressing System Medium Kit #ULTVFL05MD and V.A.C. VERALINK Cassette with Adaptor #ULTLNK0500. A 15 mm thick GranuFoam® dressing sized to 100 mm×100 by hand was placed on top of the first wound dressing sample 402. The first wound dressing sample 402, the GranuFoam® dressing, and the blood soil model in the petri dish 406A were placed into a silicon mold similar to a size of the petri dish 406A. An acrylate drape was placed on the petri dish 406A. A hole of approximately 2.5 centimeters (cm) in diameter was cut into the acrylate drape and a SENSATRAC Pad tubing with an instillation port was placed over the hole and connected to a cannister unit of a V.A.C ULTA pump system. The instillation port was then connected to a 0.9% saline solution via the V.A.C. VERALINK Cassette and the V.A.C ULTA pump.

### Instillation Cycle

V.A.C VERAFLO with instillation cycle was selected with the following settings,
a) Instillation volume was set to 10 ml and soak time of 10 minutes.
b) Target pressure was set at 125 millimeters of mercury (mmHg) with a 2 hour V.A.C time per cycle.
c) Duration of the instillation was 24 hours allowing for approximately 10-11 cycles of instillation and negative therapy.

Prior to instillation and negative therapy, imaging of the blood soil model in the petri dish 406A was captured with a hand-held digital camera. At the conclusion of the instillation and negative therapy, the drape was removed and the SENSATRAC Pad tubing disconnected. The GranuFoam® and the first wound dressing sample 402 were removed from the petri dish 406A and the remaining blood soil was allowed to air dry for at least 10 minutes. Subsequently, an image of the post-treatment blood soil model in the petri dish 406A was captured by the hand-held camera.

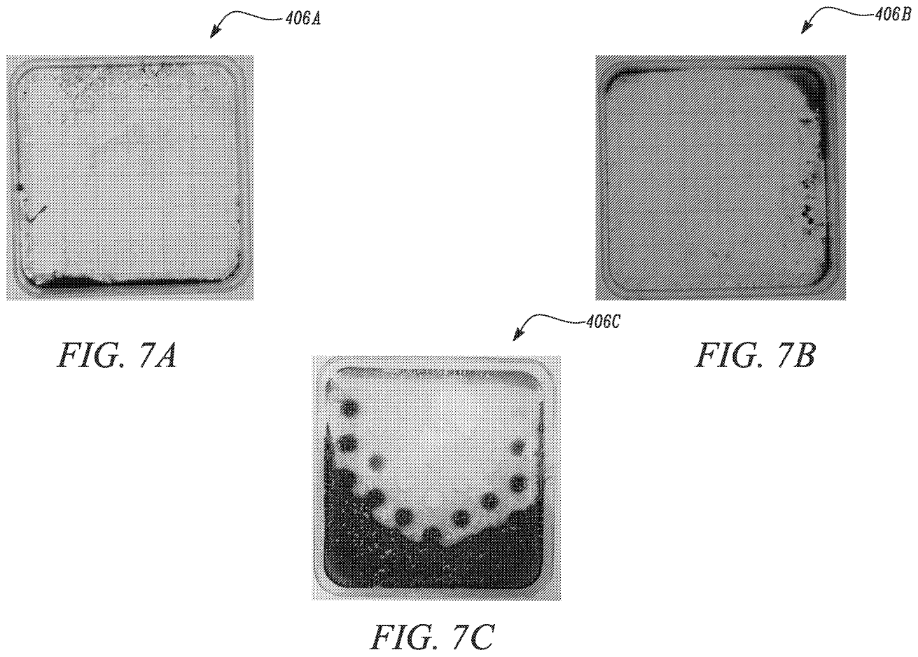

FIG. 7A illustrates a photograph of the post-treatment blood soil model in the petri dish 406A after the first wound dressing sample 402 (shown in FIG. 6A) was removed from the petri dish 406A, after the instillation and negative therapy cycle was performed thereon. FIG. 7B illustrates a photograph of the post-treatment blood soil model in the petri dish 406B after the second wound dressing sample 404 (shown in FIG. 6B) was removed from the petri dish 406B, after the instillation and negative therapy cycle was performed thereon. Based on the testing performed on the first and second wound dressing samples 402, 404 using the above steps, it was observed that the first and second wound dressing samples 402, 404 were able to transmit vacuum. It was also observed that substantially all soluble (hemoglobin) protein fractions were removed by the first and second wound dressing samples 402, 404, and no blockage issues were detected or alarms were observed during the 24 hour test. Specifically, the first and second wound dressing samples 402, 404 did not get clogged as it debrided and there were no vacuum unit alarms. Further, some fibrin layer was observed on the first and second wound dressing samples 402, 404, but no observable difference was found between the first and second wound dressing samples 402, 404. Specifically, there was no difference in a debriding power of the first and second wound dressing samples 402, 404, i.e., when used for instillation and negative therapy the relatively softer loop piles debrided just as well as the relatively stiffer loop piles.

FIG. 7C illustrates a photograph of the post-treatment blood soil model in a petri dish 406C after a conventional wound dressing (not shown) was removed from the petri dish 406C, after the instillation and negative therapy cycle was performed thereon. The conventional wound dressing was the VERAFLO Cleanse Choice. The conventional wound dressing was also tested under the same testing conditions as the first wound dressing sample 402 mentioned above. However, as visible from FIGS. 7A, 7B, and 7C, more material (i.e., the blood soil model) was removed from the first and second wound dressing samples 402, 404 as compared to the conventional wound dressing.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A wound dressing for use with a wound therapy treatment system, the wound dressing comprising:
a support layer having a first side configured to face a wound bed and a second side opposite to the first side; and
a plurality of loop piles made from a single loop material, wherein the plurality of loop piles is arranged on the first side of the support layer and not on the second side of the support layer.

2. The wound dressing of claim 1, wherein the support layer comprises a scrim, and wherein the plurality of loop piles is woven into the scrim.

3. The wound dressing of claim 1, wherein the support layer comprises a hydrophobic, open-cell foam.

4. The wound dressing of claim 1, wherein a linear mass density of the single loop material is within a range from about 50 deniers to about 2000 deniers.

5. The wound dressing of claim 1, wherein the single loop material comprises textured natural polyester yarn.

6. The wound dressing of claim 1, wherein the single loop material comprises bi-component black polyester monofilament yarn.

7. The wound dressing of claim 1, wherein the support layer comprises 500 deniers and 840 deniers multifilament natural polyester warp fibers and 2000 denier multifilament natural polyester weft fibers.

8. The wound dressing of claim 1, wherein each loop pile of the plurality of loop piles comprises about 4 ends of yarn.

9. The wound dressing of claim 1, wherein a course per inch of the plurality of loop piles is within a range from about 5 courses per inch to about 30 courses per inch.

10. The wound dressing of claim 1, wherein a wales per inch of the plurality of loop piles is within a range from about 5 courses per inch to about 30 courses per inch.

11. The wound dressing of claim 1, wherein an average loop height relative to the support layer of the plurality of loop piles is within a range from about 5 millimeters (mm) to about 10 mm.

12. The wound dressing of claim 1, wherein an average loop width between opposing portions of the plurality of loop piles measured parallel to the support layer is within a range from about 0.5 mm to about 10 mm.

13. The wound dressing of claim 1, wherein a density of the plurality of loop piles is within a range from about 50 stiches per inch to about 400 stiches per inch.

14. The wound dressing of claim 1 further comprising at least one absorbent layer adjacent to the support layer.

15. (canceled)

16. The wound dressing of claim 1, wherein the wound dressing is devoid of any other loop piles other than the plurality of loop piles.

17-21. (canceled)

22. A method of forming a wound dressing comprising:
providing a support layer having a first side configured to face a wound bed and a second side opposite to the first side; and
forming a plurality of loop piles made from a single loop material on the first side of the support layer and not on the second side of the support layer.

23. The method of claim 22, wherein forming the plurality of loop piles comprises knitting the single loop material to the first side of the support layer.

24. The method of claim 22 further comprising securing at least one free edge of the support layer.

25. The method of claim 24, wherein securing the at least one free edge of the support layer comprises at least one of impulse welding, ultrasonic welding, and serge seaming.

26. (canceled)

27. The method of claim 22, wherein the wound dressing is devoid of any other loop piles other than the plurality of loop piles.
